(19) United States
(12) Patent Application Publication
(10) Pub. No.: US 2004/0152738 A1
(43) Pub. Date: Aug. 5, 2004

(54) Pyridyl-substituted triazoles as tgf inhibitors

(76) Inventors: Laramie Mary Gaster, Harlow Essex (GB); John David Harling, Harlow Essex (GB); Jag Paul Heer, Harlow Essex (GB); Thomas Daniel Heightman, Harlow Essex (GB); Andrew Hele Payne, Harlow Essex (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/416,680

(22) PCT Filed: Nov. 15, 2001
(86) PCT No.: PCT/GB01/05036

(30) Foreign Application Priority Data
Nov. 16, 2000 (GB) 0027987.7

(51) Int. Cl. A61K 31/4439; C 07D 4 3/04

(52) U.S. Cl. 514/341; 546/268.1

(57) ABSTRACT

Pyridyl substituted triazoles of formula (I)

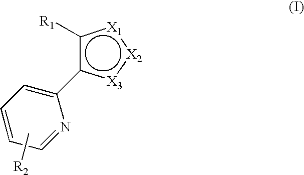

wherein R₁ is naphthyl or phenyl optionally substituted with one or more substituents selected from the group consisting of halo, —O—C₁₋₆alkyl, —S—C₁₋₆alkyl, C₁₋₆alkyl, C₁₋₆haloalkyl, —O—(CH₂)ₙ-Ph, —S—(CH₂)ₙ-Ph, cyano, phenyl, and CO₂R, wherein R is hydrogen or C₁₋₆alkyl, and n is 0, 1, 2 or 3; or R₁ is phenyl fused with an aromatic or non-aromatic cyclic ring of 5-7 members wherein said cyclic ring optionally contains up to three heteroatoms, independently selected from N, O and S, and N may be further optionally substituted by C₁₋₆ alkyl;

R₂ is H, C₁₋₆alkyl, C₁₋₆alkoxy, phenyl, NH(CH₂)ₙ-Ph, NH—C₁₋₆alkyl, halo, CN, NO₂, CONHR and SO₂NHR;

two of X₁, X₂ and X₃ are N and the other is NR₃ wherein R₃ is hydrogen, C₁₋₆alkyl, C₃₋₇cycloalkyl, —(CH₂)ₚ—CN, —(CH₂)ₚ—CO₂H, —(CH₂)ₚ—CONHR₄R₅, —(CH₂)ₚCOR₄, —(CH₂)_{q}(OR₆)₂,

—(CH₂)ₚOR₄, —(CH₂)_{q}—CH═CH—CN, —(CH₂)_{q}—CH═CH—CO₂H, —(CH₂)ₚ—CH═CH—CONHR₄R₅, (CH₂)ₚNHCOR₇ or (CH₂)ₚNR₈R₉;

R₄ and R₅ are independently hydrogen or C₁₋₆alkyl;

R₆ is C₁₋₆alkyl;

R₇ is C₁₋₇alkyl, or optionally substituted aryl, heteroaryl, arylC₁₋₆alkyl or heteroarylC₁₋₆alkyl;

R₈ and R₉ are independently selected from hydrogen, C₁₋₆alkyl, aryl and arylC₁₋₆alkyl;

p is 04; and

q is 1-4.

and salts and solvates thereof, are disclosed, as are methods for their preparation, pharmaceutical compositions containing them and their use in medicine.

This invention relates to pyridyl substituted triazoles which are inhibitors of the transforming growth factor, (“TGF”)-β signaling pathway, in particular, the phosphorylation of smad2 or smad3 by the TGF-β type I or activin-like kinase (“ALK”)-5 receptor, methods for their preparation and their use in medicine, specifically in the treatment and prevention of a disease state mediated by this pathway.

TGF-β1 is the prototypic member of a family of cytokines including the TGF-βs, activins, inhibins, bone morphogenetic proteins and Müllerian-inhibiting substance, that signal through a family of single transmembrane serine/threonine kinase receptors. These receptors can be divided in two classes, the type I or activin like kinase (ALK) receptors and type II receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The GS domain is at the amino terminal end of the intracellular kinase domain and is critical for activation by the type II receptor. Several studies have shown that TGF-β signaling requires both the ALK and type II receptors. Specifically, the type II receptor phosphorylates the GS domain of the type I receptor for TGF-β, ALK5, in the presence of TGF-β. The ALK5, in turn, phosphorylates the cytoplasmic proteins smad2 and smad3 at two carboxy terminal serines. The phosphorylated smad proteins translocate into the nucleus and activate genes that contribute to the production of extracellular matrix. Therefore, preferred compounds of this invention are selective in that they inhibit the type I receptor and thus matrix production.

Activation of the TGF-β1 axis and expansion of extracellular matrix are early and persistent contributors to the development and progression of chronic renal disease and vascular disease. Border W A., et al, N Engl. J. Med., 1994; 331(19), 1286-92. Further, TGF-β1 plays a role in the formation of fibronectin and plasminogen activator inhibitor-1, components of sclerotic deposits, through the action of smad3 phosphorylation by the TGF-β1 receptor ALK5. Zhang Y., et al, Nature, 1998; 394(6696), 909-13; Usui T., et al, Invest. Ophthalmol. Vis. Sci., 1998; 39(11), 1981-9.

Progressive fibrosis in the kidney and cardiovascular system is a major cause of suffering and death and an important contributor to the cost of health care. TGF-β1 has been implicated in many renal fibrotic disorders. Border W. A., et al, N. Engl. J. Med., 1994; 331(19), 1286-92. TGF-β1 is elevated in acute and chronic glomerulonephritis Yoshioka K., et al, Lab. Invest., 1993; 68(2), 154-63, diabetic nephropathy Yamamoto, T., et al, 1993, PNAS 90, 1814-1818., allograft rejection, HIV nephropathy and angiotensin-induced nephropathy Border W. A., et al, N. Engl. J. Med., 1994; 331(19), 1286-92. In these diseases the levels of TGF-β1 expression coincide with the production of extracellular matrix. Three lines of evidence suggest a causal relationship between TGF-β1 and the production of matrix. First, normal glomeruli, mesangial cells and non-renal cells can be induced to produce extracellular-matrix protein and inhibit protease activity by exogenous TGF-β1 in vitro. Second, neutralizing anti-bodies against TGF-β1 can prevent the accumulation of extracellular matrix in nephritic rats. Third, TGF-β1 transgenic mice or in vivo transfection of the TGF-β1 gene into normal rat kidneys resulted in the rapid development of glomerulosclerosis. Kopp J. B., et al, Lab. Invest., 1996; 74(6), 991-1003. Thus, inhibition of TGF-β1 activity is indicated as a therapeutic intervention in chronic renal disease.

TGF-β1 and its receptors are increased in injured blood vessels and are indicated in neointima formation following balloon angioplasty Saltis J., et al, Clin. Exp. Pharmacol. Physiol., 1996; 23(3), 193-200. In addition TGF-β1 is a potent stimulator of smooth muscle cell (“SMC”) migration in vitro and migration of SMC in the arterial wall is a contributing factor in the pathogenesis of atherosclerosis and restenosis. Moreover, in multivariate analysis of the endothelial cell products against total cholesterol, TGF-β1 receptor ALK5 correlated with total cholesterol (P<0.001) Blann A. D., et al, Atherosclerosis, 1996; 120(1-2), 221-6. Furthermore, SMC derived from human atherosclerotic lesions have an increased ALK5/TGF-β type II receptor ratio. Because TGF-β1 is over-expressed in fibroproliferative vascular lesions, receptor-variant cells would be allowed to grow in a slow, but uncontrolled fashion, while overproducing extracellular matrix components McCaffrey T. A., et al, Jr., J. Clin. Invest., 1995; 96(6), 2667-75. TGF-β1 was immunolocalized to non-foamy macrophages in atherosclerotic lesions where active matrix synthesis occurs, suggesting that non-foamy macrophages may participate in modulating matrix gene expression in atherosclerotic remodeling via a TGF-β-dependent mechanism. Therefore, inhibiting the action of TGF-β1 on ALK5 is also indicated in atherosclerosis and restenosis.

TGF-β is also indicated in wound repair. Neutralizing antibodies to TGF-β1 have been used in a number of models to illustrate that inhibition of TGF-β1 signaling is beneficial in restoring function after injury by limiting excessive scar formation during the healing process. For example, neutralizing antibodies to TGF-β1 and TGF-β2 reduced scar formation and improved the cytoarchitecture of the neodermis by reducing the number of monocytes and macrophages as well as decreasing dermal fibronectin and collagen deposition in rats Shah M., J. Cell. Sci., 1995, 108, 985-1002. Moreover, TGF-β antibodies also improve healing of corneal wounds in rabbits Moller-Pedersen T., Curr. Eye Res., 1998, 17, 736-747, and accelerate wound healing of gastric ulcers in the rat, Ernst H., Gut, 1996, 39, 172-175. These data strongly suggest that limiting the activity of TGF-β would be beneficial in many tissues and suggest that any disease with chronic elevation of TGF-β would benefit by inhibiting smad2 and smad3 signaling pathways.

TGF-β is also implicated in peritoneal adhesions Saed G. M., et al, Wound Repair Regeneration, 1999 November-December, 7(6), 504-510. Therefore, inhibitors of ALK5 would be beneficial in preventing peritoneal and sub-dermal fibrotic adhesions following surgical procedures.

Surprisingly, it has now been discovered that a class of 2-pyridyl substituted triazole compounds function as potent and selective non-peptide inhibitors of ALK5 kinase and therefore, have utility in the treatment and prevention of various disease states mediated by ALK5 kinase mechanisms, such as chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis and restenosis.

According to the invention there is provided a compound of formula (I) or a pharmaceutically acceptable salt thereof:

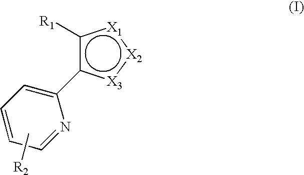

wherein R₁ is naphthyl or phenyl optionally substituted with one or more substituents selected from the group consisting of halo, —O—C₁₋₆alkyl, —S—C₁₋₆alkyl, C₁₋₆alkyl, C₁₋₆haloalkyl, —O—(CH₂)ₙ-Ph, —S—(CH₂)ₙ-Ph, cyano, phenyl, and CO₂R, wherein R is hydrogen or C₁₋₆alkyl, and n is 0, 1, 2 or 3; or R₁ is phenyl fused with an aromatic or non-aromatic cyclic ring of 5-7 members wherein said cyclic ring optionally contains up to three heteroatoms, independently selected from N, O and S and N may be further optionally substituted by C₁₋₆ alkyl;

R₂ is H, C₁₋₆alkyl, C₁₋₆alkoxy, phenyl, NH(CH₂)ₙ-Ph, NH—C₁₋₆alkyl, halo, CN, NO₂, CONHR and SO₂NHR;

Alternatively, R₂ is H, C₁₋₆alkyl, C₁₋₆alkoxy, phenyl, NH(CH₂)₂-Ph, NH—C₁₋₆alkyl, or halo.

two of X₁, X₂ and X₃ are N and the other is NR₃ wherein R₃ is hydrogen, C₁₋₆alkyl, C₃₋₇cycloalkyl, —CH₂)ₚ—CN, —(CH₂)ₚ—CO₂H, —(CH₂)ₚ—CONHR₄R₅, —(CH₂)ₚCOR₄, —(CH₂)_{q}(OR₆)₂, —(CH₂)ₚOR₄, —(CH₂)_{q}—CH═CH—CN, —(CH₂)_{q}—CH═CH—CO₂H, —(CH₂)ₚ—CH═CH—CONHR₄R₅, —(CH₂)ₚNHCOR₇ or —(CH₂)ₚNR₈R₉;

R₄ and R₅ are independently hydrogen or C₁₋₆alkyl;

R₆ is C₁₋₆alkyl;

R₇ is C₁₋₇alkyl, or optionally substituted aryl, heteroaryl, arylC₁₋₆alkyl or heteroarylC₁₋₆alkyl;

R₈ and R₉ are independently selected from hydrogen, C₁₋₆alkyl, aryl and arylC₁₋₆alkyl;

p is 0-4; and

q is 1-4.

In the triazole ring of the compounds of formula (I) it will be apparent that the double bond will be to the two unsubstituted nitrogens.

Preferably R₁ is optionally substituted naphthyl or phenyl. More preferably R₁ is phenyl optionally substituted with one or more substituents selected from halo, C₁₋₆alkoxy, C₁₋₆alkylthio, and phenyl; or R₁ is phenyl fused with an aromatic or non-aromatic cyclic ring of 5-7 members wherein said cyclic ring optionally contains up to two heteroatoms, independently selected from N, O and S, and N may be further optionally substituted by C₁₋₆ alkyl. For example R₁ represents benzo[1,3]dioxolyl, 2,3-dihydrobenzo[1,4]dioxinyl, benzoxazolyl, benzothiazolyl, benzo[1,2,5]oxadiazolyl, benzo[1,2,5]thiadiazolyl, quinoxalinyl, dihydrobenzofuranyl, benzimidazolyl, C₁₋₆ alkylbenzimidazolyl, or [1,2,4]triazolo[1,5-a]pyridyl. Alternatively, R₁ represents benzo[1,3]dioxolyl, 2,3-dihydrobenzo[1,4]dioxinyl, benzoxazolyl, benzothiazolyl, benzo[1,2,5]oxadiazolyl, benzo[1,2,5]thiadiazolyl, quinoxalinyl or dihydrobenzofuranyl. Preferably R₁ represents 4-methoxyphenyl, 3-chlorophenyl, 3-fluoro-4-methoxyphenyl or 3-chloro-4-methoxyphenyl, or R₁ represents benzo[1,2,5]thiadiazolyl, [1,2,4]triazolo[1,5-a]pyridyl, dihydrobenzofuranyl, 2,3-dihydrobenzo[1,4]dioxinyl, benzimidazolyl or C₁₋₆alkylbenzimidazolyl.

Preferably, R₂ is positioned ortho to the nitrogen of the pyridyl ring, R₂ is preferably a methyl group.

Specific compounds of the invention which may be mentioned include the following and pharmaceutically acceptable salts thereof:

5-[5-6-Methyl-pyridin-2-yl)-1H-[1,2,3]triazol-4-yl]-benzo[1,2,5]thiadiazole;

5-[2-Ethyl-5-(6-methyl-pyridin-2-yl)-2H-[1,2,3]triazol-4yl]-benzo[1,2,5]thiadiazole,

6-[5-(6-Methylpyridin-2-yl)-1H-[1,2,3]triazol-4-yl]-[1,2,4]triazolo[1,5a]pyridine,

2-[5(2,3-Dihydrobenzofuran-5-yl)-3H-[1,2,3]triazol-4-yl]-6-methylpyridine,

2-[5-2,3-Dihydrobenzo[1,4]dioxin-6-yl)-2H-[1,2,3]triazol-4-yl]-6-methylpyridine,

1-Methyl-6-[5-(6-methyl-pyridin-2-yl)-2H-[1,2,3]triazol-4-yl]-1H-benzimidazole,

6-(2-Ethyl-5-(6-methylpyridin-2-yl)-2H-[1,2,3]triazol-4-yl)-[1,2,4]triazolo[1,5-a]pyridine,

6-(2-Methyl-5-(6-methylpyridin-2-yl)-2H-[1,2,3]triazol-4-yl)-[1,2,4]triazolo[1,5-a]pyridine,

2-[5-(4-Methoxyphenyl)-2H-[1,2,3]triazol-4-yl]-6-methylpyridine,

2-[5-(3-Fluoro-4-methoxyphenyl)-2H-[1,2,3]triazol-4-yl]-6-methylpyridine and;

2-[5-(3-Chloro-4-methoxyphenyl)-2H-[1,2,3]triazol-4-yl]-6-methylpyridine.

Suitable, pharmaceutically acceptable salts of the compounds of formula (I) include, but are not limited to, salts with inorganic acids such as hydrochloride, sulfate, phosphate, diphosphate, hydrobromide, and nitrate, or salts with an organic acid such as malate, maleate, fumarate, tartrate, succinate, citrate, acetate, lactate, methanesulfonate, p-toluenesulfonate, palmitate, salicylate, and stearate.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Certain of the compounds of formula (I) may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the formula (I) or pharmaceutically acceptable derivative thereof.

The terms “C₁₋₆alkyl” and “C₁₋₇alkyl” as used herein whether on their own or as part of a larger group e.g. C₁₋₆alkoxy, mean a straight or branched chain radical of 1 to 6 and 1 to 7 carbon atoms respectively, unless the chain length is limited thereto, including, but not limited to methyl, ethyl, n-propyl, isopropyl, n-butyl, sec-butyl, isobutyl and tert-butyl.

C₁₋₆ haloalkyl groups may contain one or more halo atoms, a particular C₁₋₆ haloalkyl group that may be mentioned is CF₃.

The terms “halo” or “halogen” are used interchangeably herein to mean radicals derived from the elements chlorine, fluorine, iodine and bromine.

The term “cycloalkyl” is used herein to mean cyclic radicals, preferably of 3 to 7 carbons, including but not limited to cyclopropyl, cyclopentyl and cyclohexyl.

The term “aryl” is used herein to mean 5- to 14-membered substituted or unsubstituted aromatic ring(s) or ring systems which may include bi- or tricyclic systems, including, but not limited to phenyl, naphthyl.

The term “ALK5 inhibitor” is used herein to mean a compound, other than inhibitory smads, e.g. smad6 and smad7, which selectively inhibits the ALK5 receptor preferentially over p38 or type II receptors.

The term “ALK5 mediated disease state” is used herein to mean any disease state which is mediated (or modulated) by ALK5, for example a disease which is modulated by the inhibition of the phosphorylation of smad 2/3 in the TGF-1β signaling pathway.

The term “ulcers” is used herein to include, but not to be limited to, diabetic ulcers, chronic ulcers, gastric ulcers, and duodenal ulcers.

The compounds of formula (I) can be prepared by art-recognized procedures from known or commercially available starting materials. If the starting materials are unavailable from a commercial source, their synthesis is described herein, or they can be prepared by procedures known in the art.

Specifically, compounds of formula (I) may be prepared as illustrated in Scheme 1.

An aryl bromide is coupled with trimethylsilylacetylene using a palladium catalyst in the presence of copper(I) iodide. The trimethylsilyl group is then removed under basic conditions with potassium carbonate and the unmasked terminal acetylene is coupled to a substituted bromopyridine via palladium catalysis. The disubstituted acetylene is treated with trimethylsilylazide to afford a triazole which may be alkylated with a suitable alkylating agent, L-R₃ where L is a leaving group, e.g. I, in the presence of potassium carbonate. The resulting isomers can be separated by chromatographic methods.

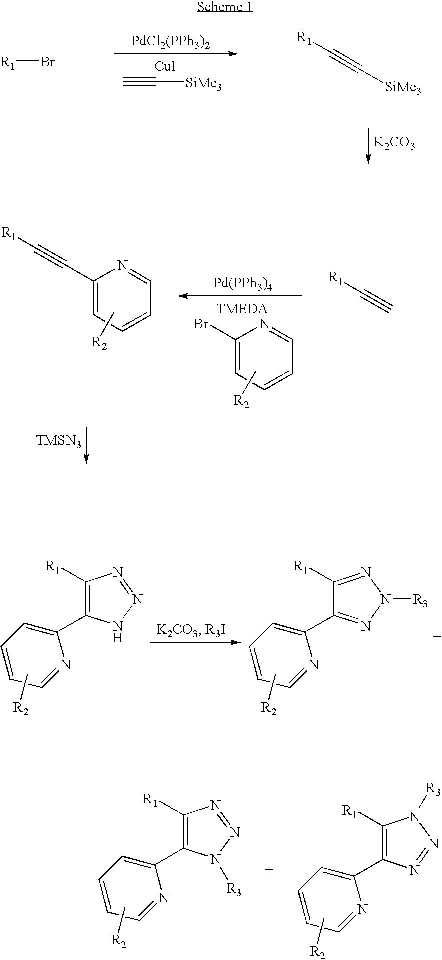

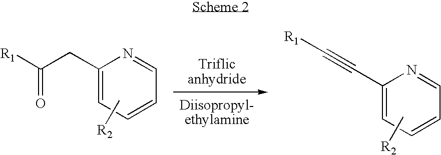

Alternatively, the disubstituted acetylene intermediates may be prepared by treatment of a suitable 1-aryl-2-pyridylethanone with Hünigs base and triflic anhydride. The 1-aryl-2-pyridylethanones may be prepared according to the procedures described in WO 00/61576.

During the synthesis of the compounds of formula (I) labile functional groups in the intermediate compounds, e.g. hydroxy, carboxy and amino groups, may be protected. A comprehensive discussion of the ways in which various labile functional groups groups may be protected and methods for cleaving the resulting protected derivatives is given in for example Protective Groups in Organic Chemistry, T. W. Greene and P. G. M. Wuts, (Wiley-Interscience, New York 2nd edition, 1991).

Further details for the preparation of compounds of formula (I) are found in the examples.

The compounds of formula (I) may be prepared singly or as compound libraries comprising at least 2, for example 5 to 1,000 compounds, and more preferably 10 to 100 compounds of formula (I). Libraries of compounds of formula (I) may be prepared by a combinatorial ‘split and mix’ approach or by multiple parallel synthesis using either solution phase or solid phase chemistry, by procedures known to those skilled in the art.

Thus according to a further aspect of the invention there is provided a compound library comprising at least 2 compounds of formula (I) or pharmaceutically acceptable salts thereof.

In another aspect of the invention there is provided the use of a compound of formula (I), or a pharmaceutically acceptable salt or solvate thereof, in therapy.

According to a further aspect of the present invention there is provided the use of a compound of formula (I) or a pharmaceutically acceptable salt thereof, in the manufacture of a medicament for the treatment of a disease mediated by the ALK5 receptor in mammals.

ALK5-mediated disease states, include, but are not limited to, chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, and restenosis.

By the term “treating” is meant either prophylactic or therapeutic therapy.

According to a further aspect of the present invention there is provided a method of inhibiting the TGF-β signaling pathway in mammals, for example, inhibiting the phosphorylation of smad2 or smad3 by the type I or activin-like kinase ALK5 receptor.

According to a further aspect of the present invention there is provided a method of inhibiting matrix formation in mammals by inhibiting the TGF-β signalling pathway, for example, inhibiting the phosphorylation of smad2 or smad3 by the type I or activin-like kinase ALK5 receptor.

The pharmaceutically effective compounds of formula (I) and pharmaceutically acceptable salts thereof, may be administered in conventional dosage forms prepared by combining a compound of formula (I) with standard pharmaceutical carriers or diluents according to conventional procedures well known in the art. These procedures may involve mixing, granulating and compressing or dissolving the ingredients as appropriate to the desired preparation.

According to a further aspect of the present invention there is provided a pharmaceutical composition comprising a compound of formula (I), or a pharmaceutically acceptable salt thereof, and a pharmaceutically acceptable carrier or diluent.

The pharmaceutical compositions of the invention may be formulated for administration by any route, and include those in a form adapted for oral, topical or parenteral administration to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

It will be recognized by one of skill in the art that the optimal quantity and spacing of individual dosages of a formula (I) compound will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of the formula (I) compound given per day for a defined number of days, can be ascertained by those skilled in the art using conventional course of treatment determination tests.

No toxicological effects are indicated when a compound of formula (I) or a pharmaceutically acceptable derivative thereof is administered in the above-mentioned dosage range.

All publications, including, but not limited to, patents and patent applications cited in this specification, are herein incorporated by reference as if each individual publication were specifically and individually indicated to be incorporated by reference herein as though fully set forth.

The following examples are to be construed as merely illustrative and not a limitation on the scope of the invention in any way. In the Examples, mass spectra were performed using an Hitachi Perkin-Elmer RMU-6E with chemical ionization technique (CI) or a Micromass Platform II instrument with electrospray (ES) ionization technique.

Abbreviations

CH₂Cl₂—dichloromethane

DMF—dimethylformamide

Et₃N—triethylamine

EtOAc—ethyl acetate

HCl—hydrochloric acid

MeOH—methanol

MgSO₄—magnesium sulphate

Na₂SO₄—sodium sulphate

THF—tetrahydrofuran

TMS—trimethylsilyl

TMEDA—N,N,N′,N′-tetramethylethylenediamine

### EXAMPLES

### Example 1

### 5-[5-(6-Methyl-pyridin-2-yl)1H-[1,2,3]triazol-4-yl]-benzo[1,2,5]thiadiazole

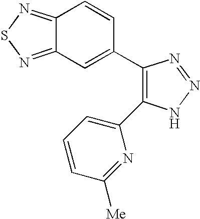

5-(6-Methyl-pyridin-2-ylethynyl)-benzo[1,2,5]thiadiazole (300 mg, 1.2 mmol) was dissolved in DMF (50 ml) to which was added trimethylsilylazide (415 mg, 3.6 mmol). The reaction mixture was heated at reflux under argon at 130° C. overnight. The DMF was removed under reduced pressure. The residue was partitioned between ethyl acetate and water. The ethyl acetate layer was collected, dried and concentrated under reduced pressure. The title compound was purified on an SCX column eluting with methanolic ammonia (265 mg, 75%); ¹H NMR (250 MHz, CDCl₃)δ: 2.62 (3H, s), 7.32-7.44 (3H, multiplet), 7.63 (1H, t, J=7.7 Hz), 7.76 (1H, d, J=7.7 Hz), 7.99 (1H, d, J=9 Hz), NH not observed; m/z (API⁺): 295 (MH⁺).

### Example 2

### 5-[2-Ethyl-5-(6-methyl-pyridin-2-yl)-2H-[1,2,3]triazol-4-yl]-benzo[1,2,5]-thiadiazole

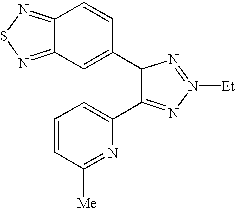

5-[5-(6-Methyl-pyridin-2-yl)-2H-[1,2,3]triazol-4yl]-benzo[1,2,5]thiadiazole (120 mg, 0.41 mmol) was dissolved in DMF (10 ml) and treated with potassium carbonate (85 mg, 0.61 mmol) and iodoethane (49 μL, 0.61 mmol). The reaction mixture was stirred at room temperature for 18 h then evaporated to dryness under reduced pressure. The residue was partitioned between water and ethyl acetate. The organic phase was dried (MgSO₄) and evaporated to dryness under reduced pressure. The title compound was isolated by silica gel column chromatography using ethyl acetate as eluent (40 mg, 30%); ¹H NMR (400 MHz, CDCl₃) δ: 1.69 (3H, t, J=8 Hz), 2.56 (3H, s), 4.61 (2H, q, J=8 Hz), 7.17 (1H, d, J=7.5 Hz), 7.52 (1H, d, J=7.5 Hz), 7.63 (1H, t, J=7.5 Hz), 7.97 (1H, d, J=9.0 Hz), 8.03 (1H, d, J=9.0 Hz), 8.51 (1H, s); m/z (API⁺): 323 (MH⁺).

### Example 3

### 6-[5-(6-Methylpyridin-2-yl)-1H-[1,2,3]triazol-4-yl]-[1,2,4]triazolo[1,5a]pyridine

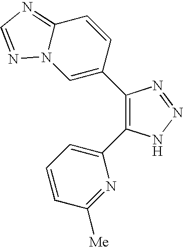

b 6-(6-Methylpyridin-2-ylethynyl)-[1,2,4] triazolo[1,5-a]pyridine(200 mg, 0.85 mmol, 1 eq) prepared according to Preparation 4 from 6-ethynyl-[1,2,4]triazolo[1,5-a]pyridine was dissolved in anhydrous DMF (1.5 ml) under argon. To this was added trimethylsilylacetylene azide (0.4 ml, 0.34 g, 3 mmol, 3.5 eq) and the solution refluxed at 130° C. for 16 h. After removing the solvents, the residue was taken up in water (30 ml) and extracted with ethyl acetate (2×30 ml). The organic layers were combined, washed with water and brine (20 ml of each), dried (MgSO₄) and the solvent removed. The crude product was purified by using a 2 ml SCX acid ion-exchange column, which was eluted with dichloromethane then 1:1 dichloromethane: methanol and finally 3% ammonia in methanol (20 ml) to remove the basic product, yielding an orange solid product (25 mg, 10%). The hydrochloride salt of this compound was made by dissolution of the solid in the minimum quantity of methanol, the addition of 1M HCl in ether, dilution with ethyl acetate and removal of the solvent to yield an orange solid; ¹H NMR (400 MHz, CDCl₃) δ: 9.61 (1H, b.s), 8.40 (1H, s), 8.04 (1H, d), 7.80 (1H, d), 7.66 (2H, m), 7.20 (1H, d), 2.61 (3H, s), Nh not observed; m/z [APCIMS]: 276 [M−H]⁻, 278 [M+H]⁺.

### Example 4

### 2-[5-(2,3-Dihydrobenzofuran-5-yl)-3H-[1,2,3]triazol-4yl]-6-methylpyridine

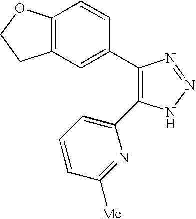

Prepared from 2-(2,3-dihydrobenzofuran-5-ylethynyl)-6-methylpyridine according to the procedure for Example 3. ¹H NMR (250 MHz, CDCl₃)δ: 7.56 (2H, m), 7.42 (1H, s), 7.39 (1H, dd), 7.12 (1H, d), 6.82 (1H, d), 4.63 (2H, t), 3.25 (2H, t), 2.60 (3H, s), NH not observed; m/z: 277.1 [M−H]⁻, 279 [M+H]⁺

### Example 5

### 2-[5-(2,3-Dihydrobenzo[1,4]dioxin-6-yl)-2H-[1,2,3]triazol-4-yl]-6-methyl-pyridine

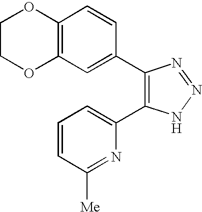

To a stirred solution of the 2-(2,3-dihydro-benzo[1,4]dioxin-6-ylethynyl)-6-methylpyridine (prepared from 1-(2,3-dihydro-benzo[1,4]dioxin-6-yl)-2-(6-methylpyridin-2-yl)ethanone according to the procedure of Preparation 9) (0.830 g, 3.30 mmol, 1.0 eq) in 7 ml dry DMF at R.T. was added azidotrimethylsilane (1.35 ml, 1.14 g, 9.90 mmol, 3.0 eq). The solution was heated at 65° C. under argon for a total of 5 days, 1 ml more azidotrimethylsilane being added after 3 days. The reaction mixture was allowed to cool and the DMF removed by distillation under reduced pressure. The residue was partitioned between water and EtOAc and the layers separated. The organic phase was washed with brine and dried over MgSO₄. Concentration to an oil, followed by flash column chromatography over silica, eluting with CH₂Cl₂:MeOH:Et₃N 95:4:1, afforded the title compound as a thick oil, 0.649 g (67%). ¹H NMR (250 MHz; CDCl₃) δ: 7.57 (1H, t), 7.44 (1H, d), 7.27 (2H, m), 7.15 (1H, t), 6.89 (1H, d), 4.32-4.25 (4H, m), 2.59 (3H, s), NH not observed; m/z [ESMS]: 295.1 [M+H]⁺.

### Example 6

### 1-Methyl-6-[5-(6-methylpyridin-2-yl)-2H-[1,2,3]triazol-4-yl]-1H-benzimidazole

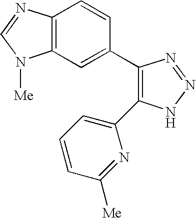

Prepared from 1-methyl-6-(6-methyl-pyridin-2-yl-ethynyl)-1H-benzimidazole according to the procedure for Example 3. ¹H NMR (400 MHz, CDCl₃)δ: 7.94 (2H, d, J=8 Hz), 7.81 (1H, d, J=13 Hz), 7.53 (2H, m), 7.43 (1H, d, J=12 Hz), 7.13 (1H, d, J=12 Hz), 3.83 (3H, s), 2.59 (3H, s); m/z(API⁺): 291(MH⁺).

### Example 7

### 6-(2-Ethyl-5-(6-methylpyridin-2-yl)-2H-[1,2,3]triazol-4-yl)-[1,2,4]triazolo[1,5-a]pyridine

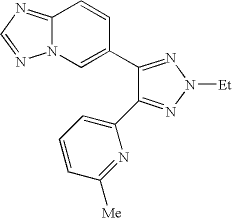

Prepared from 6-[5-(6-methylpyridin-2-yl)-1H-[1,2,3]triazol-4-yl]-[1,2,4] triazolo[1,5-a]pyridine according to the procedure for Example 2. ¹H NMR (400 MHz, CDCl₃) δ: 9.72 (1H, s), 8.37 (1H, s), 8.07 (1H, d), 7.75 (1H, d), 7.67 (2H, m), 7.18 (1H, t), 4.59 (2H, q), 2.60, (3H, s), 1.68 (3H, t); m/z [APCIMS]: 306.3 [M+H]⁺

### Example 8

### 6-(2-Methyl-5-(6-methylpyridin-2-yl)-2H-[1,2,3]triazol-4-yl)-[1,2,4]triazolo[1,5-a]pyridine

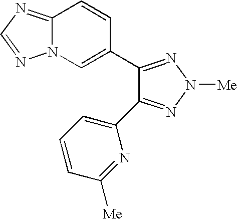

Prepared from 6-[5-(6-methylpyridin-2-yl)-1H-[1,2,3]triazol-4-yl]-[1,2,4] triazolo[1,5-a]pyridine according to the procedure for Example 2 using iodomethane as the alkylating agent. ¹H NMR (400 MHz, CDCl₃)δ: 9.71 (1H, s), 8.37 (1H, s), 8.05 (1H, dd), 7.75 (1H, d), 7.67 (2H, m), 7.18 (1H, dd), 4.32 (3H, s), 2.61, (3H, s); m/z [APCIMS]: 292.2 [M+H]⁺

### Example 9

### 2-[5-(4-Methoxyphenyl)-2H-[1,2,3]triazol-4-yl]-6-methylpyridine

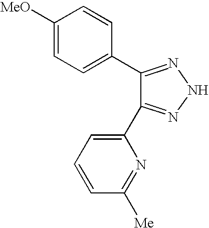

Prepared from 2-(4-methoxyphenylethynyl)-6-methylpyridine according to the procedure for Example 3. ¹H NMR (400 MHz, CDCl₃): 7.84 (1H, t), 7.50 (2H, m), 7.30 (1H, d), 6.95 (2H, m), 3.85 (3H, s), 2.79 (3H, s); m/z [APCIMS]: 267.2 [M+H]⁺, 265.1 [M−H⁺]

### Example 11

### 2-[5-3-Fluoro-4-methoxyphenyl)-2H-[1,2,3]triazol-4-yl]-6-methylpyridine

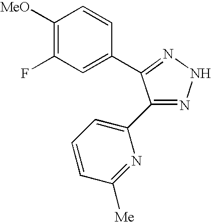

Prepared from 2-(3-fluoro-4-methoxyphenylethynyl)-6-methylpyridine (300 mg, 1.1 mmol, 1 eq) according to the procedure for Example 3. ¹H NMR (250 MHz, CDCl₃) δ: 7.55 (2H, m), 7.46 (2H, m), 7.15 (1H, d), 6.99 (1H, t), 3.94 (3H, s), 2.59 (3H, s), NH not observed.

### Example 12

### 2-[5-(3-Chloro-4methoxyphenyl)-2H-[1,2,3]triazol-4-yl]-6-methylpyridine

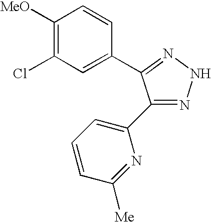

Prepared from 2-(3-chloro-4-methoxyphenylethynyl)-6-methylpyridine according to the procedure for Example 3. ¹H NMR (400 MHz, CDCl₃) δ: 7.92 (1H, t), 7.63 (1H, d), 7.54 (2H, m), 7.30 (1H, d), 6.94 (1H, d), 3.95 (3H, s), 2.75 (3H, s), NH not observed.

Preparation 1: 5-Bromo-benzo[1,2,5]thiadiazole

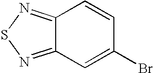

To 4-bromo-benzene-1,2-diamine (17 g, 91 mmol) was added thionyl chloride (200 ml). One drop of DMF was added to the reaction mixture. The reaction mixture was heated at reflux under argon at 80° C. overnight. The reaction mixture was cooled to room temperature and added portionwise to ice in a large beaker and neutralised with solid sodium bicarbonate. The mixture was partitioned between ethyl acetate and water. The ethyl acetate layer was collected and dried (MgSO₄). The solvent was removed under reduced pressure. The title compound was isolated by column chromatography on silica gel eluting with 90% ethyl acetate/10% methanol. (12 g, 62%); ¹H NMR (250 MHz, CDCl₃) δ: 7.61 (1H, dd, J=9, 2 Hz), 7.82 (1H, d, J=9 Hz), 8.16 (1H, s).

Preparation 2: 5-Trimethylsilanylethynylbenzo[1,2,5]thiadiazole

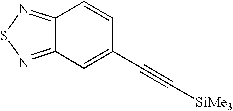

A solution of 5-bromo-benzo[1,2,5]thiadiazole (10 g, 80 mmol) was degassed with argon for 10 min, then copper iodide (1.53 g, 8 mmol) and bis-triphenylphospine-palladium-dichloride (1.12 g, 1.6 mmol) were added. TMS-acetylene (12.75 ml) was added via syringe followed by dropwise addition of diisopropylamine over 20 min. The reaction mixture was stirred at ambient temprature overnight. The THF was removed under reduced pressure. Ethyl acetate was added to the resulting brown oil and the mixture was filtered through celite. The residue was partitioned between ethyl acetate and water. The title compound was isolated by column chromatography using petroleum ether as the eluent (9 g, 49%); m/z (API⁺): 231.1 (MH⁺), m/z (API⁻): 233.1 (M).

Preparation 3: 5-Ethynyl-benzo[1,2,5]thiadiazole

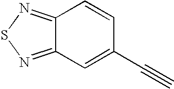

To a stirred solution of 5-trimethylsilanylethynyl-benzo[1,2,5]thiadiazole (9 g) in methanol (200 ml) was added potassium carbonate (16 g). After stirring at ambient temperature for 1 h, the reaction mixture was filtered and evaporated to dryness. The residue was partitioned between ethyl acetate and water. The ethyl acetate layer was collected, dried (Na₂SO₄) and evaporated to dryness under reduced pressure to afford the title compound (6 g, 96%); m/z (API⁺): 160 (M⁺).

Preparation 4: 5-(6Methyl-pyridin-2-ylethynyl)-benzo[1,2,5]thiadiazole

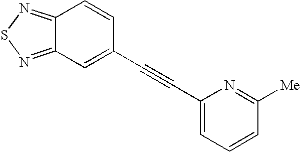

5-Ethynyl-benzo[1,2,5]thiadiazole (6 g, 38 mmol), 2-bromo-6-methyl-pyridine (13 g, 76 mmol) and copper iodide (0.7 g, 3.8 mmol) were dissolved in tetrahydrofuran (100 ml) TMEDA (100 ml). The mixture was degassed with argon for 10 min followed by addition of tetrakis(triphenylphosphine)palladium(0) (2.2 g, 1.9 mmol). The reaction was heated at 50° C. for 5 h. The solvent was removed under reduced pressure. The residue was partitioned between ethyl acetate/water. The organic layer was dried (Na₂SO₄). The title compound was isolated by column chromatography on silica gel using 3:1 petroleum ether/ethyl acetate as the eluent (5.2 g, 55%); ¹H NMR (250 MHz, CDCl₃)δ: 2.61 (3H, s), 7.3 (2H, d, J=8 Hz), 7.58 (1H, t, J=7.73 Hz), 7.72 (1H, d, J=9 Hz), 7.97 (1H, d, J=9 Hz), 8.25 (1H, s); m/z (API⁺): 252 (MH⁺).

Preparation 5: N′-(5-Bromo-2-aminopyridine)-N,N-dimethylformamidine

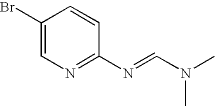

5-Bromo-2-aminopyridine (9.8 g, 56.6 mmol, 1 eq) was dissolved in dry DMF (20 ml) and dry dimethylformamide dimethylacetal (20 ml) under argon. The solution was refluxed at 130° C. for 16 h, allowed to cool, and the solvents removed. The resultant residue was used in the next stage without purification; m/z [APCIMS]: 228.0/230.0 [M+H]⁺.

Preparation 6: 6-Bromo-[1,2,4] triazolo[1,5-a]pyridine

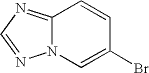

N′-(5-bromo-2-aminopyridine)-N,N-dimethylformamidine (16.2 g, ˜56.6 mmol, 1 eq) was dissolved in methanol (90 ml) and pyridine (10 ml) under argon and cooled to 0° C. To this was added, with stirring, hydroxylamine-O-sulfonic acid (7.3 g, 75.2 mmol, 1.3 eq) to form a purple suspension. This was allowed to reach room temperature and stirred for 16 h. After removing the solvents, the residue was suspended in aqueous sodium hydrogen carbonate (200 ml) and extracted with ethyl acetate (2×200 ml). The organic layer was then washed with water and brine (100 ml of each), dried (MgSO₄) and the solvent removed. Purification by flash chromatography on silica, eluting with a gradient solvent system of first 2:1 40-60° C. petroleum ether:ethyl acetate to 1:1 40-60° C. petroleum ether:ethyl acetate afforded the product as a pale yellow solid (5 g, 44.6%); ¹H NMR (250 MHz, CDCl₃) δ: 8.77 (1H, s), 8.34 (1H, s), 7.69 (1H, d), 7.65 (1H, d); m/z [APCIMS]: 198.0/200.0 [M+H]⁺.

Preparation 7: 2,3-Dihydrobenzofuran-5-carboxylic acid methoxy methyl amide

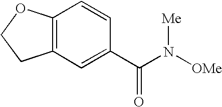

2,3-Dihydrobenzofuran-5-carboxylic acid (5 g, 30 mmol, 1 eq) was dissolved in dichloromethane (150 ml) under argon. To this was added oxalyl chloride (3.4 ml, 4.95 g, 39 mmol, 1.3 eq), followed by the dropwise addition of several drops of DMF resulting in the effervesence of a large quantity of gas. This was allowed to stir for three hours, after which time gas evolution had ceased and a pale solution had formed. To this was added N,O-dimethylhydroxylamine hydrochloride (4.1 g, 42 mmol, 1.4 eq), followed by the dropwise addition of triethylamine (12.57 ml, 9.11 g, 90 mmol, 3 eq), resulting in the formation of a large quantity of white solid (Et₃NHCl). The solution was allowed to stir overnight, before being extracted with saturated aqueous sodium bicarbonate (150 ml). The aqueous layer was back-extracted with dichloromethane (50 ml) and the organic layers combined. The organic layers were washed with water and brine (100 ml of each), dried over anhydrous MgSO₄, and the solvent removed. The crude residue was purified by flash column chromatography eluting with 3:1 40-60° petroleum ether: ethyl acetate to give an off-white solid (4.5 g, 72%) ¹H NMR (250 MHz, CDCl₃)δ: 7.61 (1H, d), 7.56 (1H, dd), 6.79 (1H, d), 4.62 (2H, t), 3.57 (3H, s), 3.49 (3H, s), 3.24 (2H, t); m/z [APCIMS]: 208.1 [M+H]⁺

Preparation 8: 1-(2,3-Dihydrobenzofuran-5-yl)-2-(6-methylpyridin-2-yl)ethanone

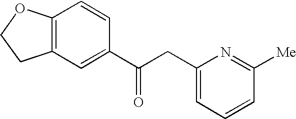

2,6-Dimethylpyridine (1.24 ml, 1.14 g, 10.62 mmol, 1.1 eq) was dissolved in THF (40 ml) under argon, and cooled to −30° C. To this was added dropwise n-butyl lithium (2.5 M in hexane, 4.25 ml, 10.62 mmol, 1.1 eq) resulting in the formation of a dark red solution. This was left to stir at −30° C. for 1 h. 2,3-Dihydrobenzofuran-5-carboxylic acid methoxy methyl amide (2.0 g, 9.65 mmol, 1 eq) was dissolved in THF (10 ml) under argon and cooled to −30° C. To this was added the cold anion solution in small portions via cannular needle. The resulting pink solution was stirred at −30° C. for twenty minutes before being allowed to slowly warm up to room temperature and left to stir overnight. The solvent was removed and the residue taken up in saturated aqueous ammonium chloride solution (100 ml) which was then extracted with ethyl acetate (2×100 ml). The organic layers were combined, washed with water, brine (100 ml of each), dried over anhydrous MgSO₄, and the solvent removed. The crude was purified by flash column chromatography, eluting with 1:1 40-60° C. petroleum ether: ethyl acetate to yield a flourescent yellow product (1.3 g, 53%). ¹H NMR (400 MHz, CDCl₃) δ: 7.95 (1H, s), 7.92 (1H, d), 7.66 (1H, m), 7.08 (1H, d), 7.02 (1H, d), 6.79 (1H, d), 4.63 (2H, t), 4.4 (2H, s), 3.24 (2H, t), 2.54 (3H, s); m/z [APCIMS]: 252.1 [M−H]⁻, 254.2 [M+H]⁺

Preparation 9: 2-(2,3-Dihydrobenzofuran-5-yl ethynyl)-6-methylpyridine

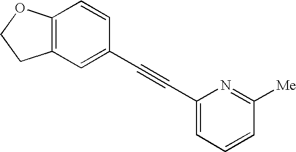

1-(2,3-Dihydrobenzofuran-5-yl)-2-(6-methylpyridin-2-yl)ethanone (200 mg, 0.79 mmol, 1 eq) was dissolved in dichloromethane (1.5 ml) under argon. To this was added Hünig's base (0.7 ml, 0.5 g, 3.9 mmol, 5 eq) followed by the dropwise addition of triflic anhydride (0.16 ml, 0.27 g, 0.95 mmol, 1.2 eq). This resulted in the formation of a brown solution that was left to stir overnight. The reaction mixture was extracted with saturated aqueous sodium bicarbonate solution (5 ml), and the aqueous layer was back extracted with dichloromethane (5 ml). The organic layers were combined, washed with water and brine (5 ml of each), dried over anhydrous MgSO₄, and the solvent removed. The crude residue was columned in 3:1 40-60° C. petroleum ether: ethyl acetate to yield a pale yellow solid (160 mg, 80%). ¹H NMR (400 MHz, CDCl₃) δ:7.54 (1H, t), 7.43 (1H, s), 7.39 (1H, dd), 7.37 (1H, d), 7.07 (1H, d), 6.75 (1H, d), 4.60 (2H, t), 3.21 (2H, t), 2.58 (3H, s); m/z [APCIMS]: 236.2 [M+H]⁺

Biological Data

The biological activity of the compounds of the invention may be assessed using the following assays:

Method for Evaluating AJLK5 Kinase Phosphorylation of smad3

Basic Flash-Plates (NEN Life Sciences) were coated by pipetting 100 micro liter of 0.1 molar sodium bicarbonate (pH 7.6), containing 150 nanograms of the fusion protein glutathion-S-transferase-smad3/100 micro liter of coating buffer. Plates were covered and incubated at room temperature for 10-24 hours. Then the plates were washed 2 times with 200 micro liter of coating buffer (0.1 molar sodium bicarbonate) and allowed to air dry for 24 hours.

For the phosphorylation reaction each well received 90 microliter containing 50 millimolar HEPES buffer (pH 7.4); 5 millimolar MgCl₂; 1 millimolar CaCl₂; 1 millimolar dithiothreitol; 100 micromolar guanosine triphosphate; 0.5 micro Ci/well gamma³³P-adenosine triphosphate (NEN Life Sciences) and 400 nanograms of a fusion protein of glutathion —S-transferase at the N-terminal end of the kinase domain of ALK5 (GST-ALK5). Background counts were measured by not adding any GST-ALK5. Inhibitors of ALK5 were evaluated by determining the activity of the enzyme in the presence of various compounds. Plates were incubated for 3 hours at 30° C. After incubation the assay buffer was removed by aspiration and the wells were washed 3 times with 200 microliter cold 10 millimolar sodium pyrophosphate in phosphate buffered saline. The last wash was aspirated and blotted plate dry. Plate was then counted on a Packard TopCount.

Inhibition of Matrix Markers: Western Blot Protocol

Data confirming activity in the enzyme assay was obtained as follows:

Cells were grown to near confluence in flasks, starved overnight and treated with TGF-beta and compounds. Cells were washed at 24 or 48 hours after treatment with ice cold phosphate buffered saline, then 500 microliter of 2× loading buffer was added to plate and cells were scraped and collected in microcentrifuge tube. (2× loading buffer: 100 mM Tris-Cl, pH6.8, 4% sodium dodecyl sulfate, 0.2% bromophenol blue, 20% glycerol, 5% beta-mercapto-ethanol). Cells were lysed in tube and vortexed. Sample was boiled for 10 minutes. 20 microliters of sample was loaded on 7.5% polyacrylamide gel (BioRad) and electrophoresed.

Size fractionated proteins in gel were transferred to nitrocellulose membrane by semidry blotting. Membrane was blocked overnight with 5% powdered milk in phosphate buffer saline (PBS) and 0.05% Tween-20 at 4 degrees C. After 3 washes with PBS/Tween membranes were incubated with primary antibody for 4 hours at room temperature. After three washes with PBS/Tween membrane was incubated with secondary antibody for 1 hour at room temperature. Finally, a signal was visualized with ECL detection kit from Amersham.

The compounds of this invention generally show ALK5 receptor modulator activity having IC₅₀ values in the range of 0.0001 to 10 μM.

1. A compound of formula (I) or a pharmaceutically acceptable salt thereof: 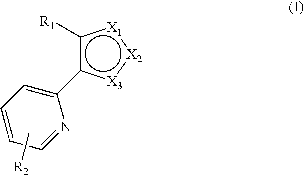
wherein R₁ is naphthyl or phenyl optionally substituted with one or more substituents selected from the group consisting of halo, —O—C₁₋₆alkyl, —S—C₁₋₆alkyl, C₁₋₆alkyl, C₁₋₆haloalkyl, —O—(CH₂)ₙ-Ph, —S—(CH₂)ₙ-Ph, cyano, phenyl, and CO₂R, wherein R is hydrogen or C₁₋₆alkyl, and n is 0, 1, 2 or 3; or R₁ is phenyl fused with an aromatic or non-aromatic cyclic ring of 5-7 members wherein said cyclic ring optionally contains up to three heteroatoms, independently selected from N, O and S and N may be further optionally substituted by C₁₋₆ alkyl;
R₂ is H, C₁₋₆alkyl, C₁₋₆alkoxy, phenyl, NH(CH₂)ₙ-Ph, NH-C₁₋₆alkyl, halo, CN, NO₂, CONHR and SO₂NHR;
two of X₁, X₂ and X₃ are N and the other is NR₃ wherein R₃ is hydrogen, C₁₋₆alkyl, C₃cycloalkyl, —(CH₂)ₚ—CN, —(CH₂)ₚ—CO₂H, —(CH₂)ₚ—CONHR₄R₅, —(CH₂)ₚCOR₄, —(CH₂)_{q}(OR₆)₂,
—(CH₂)ₚOR₄, —(CH₂)_{q}—CH═CH—CN, —(CH₂)_{q}—CH═CH—CO₂H, —(CH₂)ₚ—CH═CH—CONHR₄R₅, (CH₂)ₚNHCOR₇ or (CH₂)ₚNR₈R₉;
R₄ and R₅ are independently hydrogen or C₁₋₆alkyl;
R₆ is C₁₋₆alkyl;
R₇ is C₁₋₇alkyl, or optionally substituted aryl, heteroaryl, arylC₁₋₆alkyl or heteroarylC₁₋₆alkyl;
R₈ and R₉ are independently selected from hydrogen, C₁₋₆alkyl, aryl and arylC₁₋₆alkyl;
p is 0-4; and
q is 1-4.

2. A compound according to claim 1 wherein R₁ is phenyl optionally substituted with one or more substituents selected from halo, C₁₋₆alkoxy, C₁₋₆alkylthio, and phenyl; or R₁ is phenyl fused with an aromatic or non-aromatic cyclic ring of 5-7 members wherein said cyclic ring optionally contains up to two heteroatoms, independently selected from N, O and S, and N may be further optionally substituted by C₁₋₆alkyl.

3. A compound according to claim 2 wherein R₁ represents 4-methoxyphenyl, 3-chlorophenyl, 3-fluoro-4-methoxyphenyl or 3-chloro-4-methoxyphenyl, or R₁ represents benzo[1,2,5]thiadiazolyl, [1,2,4]triazolo[1,5-a]pyridyl, dihydrobenzofuranyl, 2,3-dihydrobenzo[1,4]dioxinyl, benzimidazolyl or C₁₋₆alkylbenzimidazolyl.

4. A compound according to any one of claims 1 to 3 wherein R₂ is positioned ortho to the nitrogen of the pyridyl ring.

5. A compound according to claim 4 wherein R₂ is methyl.

6. A compound according to claim 1 selected from:
5-[5-(6-Methylpyridin-2-yl)-1H-[1,2,3]triazol-4-yl]-benzo[1,2,5]thiadiazole;
5-[2-Ethyl-5-(6-methylpyridin-2-yl)-2H-[1,2,3]triazol-4-yl]-benzo[1,2,5]thiadiazole,
6-[5-(6-Methylpyridin-2-yl)-1H-[1,2,3]triazol-4-yl]-[1,2,4]triazolo[1,5-a]pyridine,
2-[5-(2,3-Dihydrobenzofuran-5-yl)-3H-[1,2,3]triazol-4-yl]-6-methylpyridine,
2-[5-(2,3-Dihydrobenzo[1,4]dioxin-6-yl)-2H-[1,2,3]triazol-4-yl]-6-methylpyridine,
1-Methyl-6-[5-(6-methylpyridin-2-yl)-2H-[1,2,3]triazol-4-yl]-1H-benzimidazole,
6-(2-Ethyl-5-(6-methylpyridin-2-yl)-2H-[1,2,3]triazol-4-yl)-[1,2,4]triazolo[1,5-a]pyridine
6-(2-Methyl-5-(6-methylpyridin-2-yl)-2H-[1,2,3]triazol-4-yl)-[1,2,4]triazolo[1,5-a]pyridine;
2-[5-(4-Methoxyphenyl)-2H-[1,2,3]triazol-4-yl]-6-methylpyridine,
2-[5-(3-Fluoro-4-methoxyphenyl)-2H-[1,2,3]triazol-4-yl]-6-methylpyridine,
2-[5-(3-Chloro-4-methoxyphenyl)-2H-[1,2,3]triazol-4-yl]-6-methylpyridine
and pharmaceutically acceptable salts thereof.

7. A pharmaceutical composition comprising a compound according to any one of the claims 1 to 6, or a pharmaceutically acceptable salt thereof, and a pharmaceutically acceptable carrier or diluent.

8. The use of a compound of formula (I) as claimed in any one of claims 1 to 6, or a pharmaceutically acceptable salt or solvate thereof, in therapy.

9. The use of a compound of formula (I) as claimed in any one of claims 1 to 6, or a pharmaceutically acceptable salt thereof, in the manufacture of a medicament for the treatment of a disease mediated by the ALK5 receptor in mammals.

10. A method of inhibiting the TGF-β signaling pathway in mammals, comprising administering to a mammal, comprising administering to a mammal in need of such treatment, a therapeutically effective amount of a compound according to any one of claims 1 to 6, or a pharmaceutically acceptable salt thereof.

11. A method for treating a disease selected from chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, and restenosis, comprising administering to a mammal in need of such treatment, a therapeutically effective amount of a compound according to any one of claims 1 to 6, or a pharmaceutically acceptable salt thereof.

12. A method for inhibiting matrix formation in mammals, comprising administering to a mammal, a therapeutically effective amount of a compound according to any one of claims 1 to 6, or a pharmaceutically acceptable salt thereof.
